(19) United States
(12) Patent Application Publication
(10) Pub. No.: US 2022/0093270 A1
(43) Pub. Date: Mar. 24, 2022

(54) Few-Shot Learning and Machine-Learned Model for Disease Classification

(71) Applicant: Siemens Healthcare GmbH, Erlangen (DE)

(72) Inventors: Andrei Bogdan Gheorghita, Princeton, NJ (US); Costin Florian Ciusdel, Plainsboro, NJ (US); Lucian Mihai Itu, Plainsboro, NJ (US); Teodora Chitiboi, Jersey City, NJ (US); Puneet Sharma, Princeton Junction, NJ (US)

(21) Appl. No.: 17/301,397

(22) Filed: Apr. 1, 2021

Related U.S. Application Data
(60) Provisional application No. 63/080,805, filed on Sep. 21, 2020.

(30) Foreign Application Priority Data
Sep. 21, 2020 (EP) 20465560.9

(51) Int. Cl. G16H 50/70; G06K 9/62; G06N 3/08; G06N 3/04

(52) U.S. Cl. CPC G16H 50/70; G06K 9/6268; G06N 3/0454; G06N 3/08; G06K 9/6256

(57) ABSTRACT

A machine-learned model classifies disease, such as a CVD type or sub-type. After identifying a link between the pathology (e.g., CVD type or sub-type) and one or more functional and/or anatomical characteristics, machine learning is performed to learn to predict the functional and/or anatomical characteristics from medical data. The trained model is then adapted using few-shot learning to predict the class of disease. As a result of this few-shot learning approach, less training data may be needed for disease classification. A greater number of classifiers trained to classify a greater number of diseases may be created. The machine-trained classifier(s) is applied to medical data of a patient to diagnose that patient and/or for clinical decision support

### RELATED APPLICATION

The present patent document claims the benefit of the filing date under 35 U.S.C. § 119(e) of Provisional U.S. Patent Application Ser. No. 63/080,805, filed Sep. 21, 2020 and claims the benefit of EP 20465560.9, filed on Sep. 21, 2020, which are incorporated herein by reference.

### BACKGROUND

The present embodiments relate to disease classification using a machine-learned model. One example is for cardiovascular disease (CVD). While CVDs are the main cause of death worldwide, there is also a very large number of CVD types and subtypes. CVD classification can vary between regions, countries, and continents. Hence, developing reliable methods and algorithms for diagnosing and classifying all different variants of CVD is not feasible.

Each CVD type or subtype is distinguishable by a set of anatomical and functional characteristics that can be identified in medical data (imaging and non-imaging). For example, a wide range of pathologies like cardiomyopathy, coronary artery disease, myocardial infarction, heart valve disease, and systolic heart failure are characterized by a reduced ejection fraction (EF), a functional characteristic. As another example, coronary artery disease is characterized by reduced LV wall motion (anatomical characteristic), and a reduced fractional flow reserve (FFR), a functional characteristic. Based on these characteristics, CVD diagnosis and classification are performed routinely by clinical experts, following years of training and clinical practice.

Deep learning (DL) learns patterns in medical images, providing for clinical decision-making processes, like CVD diagnosis and classification. While numerous examples of DL-based automated disease diagnosis and classification methods can be found in literature, these address only a very small subset of CVD types and subtypes. One reason for this is that the development of reliable and accurate DL models requires large training databases. However, it is very challenging, time-consuming and costly to collect such large datasets. Moreover, for some CVDs, the prevalence in the general population is still relatively low, which means in practice that even for a large specialized clinical center it would take several years to assemble a dataset in the order of thousands needed for training DL-based automatic disease classification.

### SUMMARY

Systems, methods, and instructions on computer readable media are provided for machine training for and classification with a machine-learned model of disease, such as a CVD type or sub-type. After identifying a link between the pathology (e.g., CVD type or sub-type) and one or more functional and/or anatomical characteristics, machine learning is performed to learn to predict the functional and/or anatomical characteristics from medical data. The trained model is then adapted using few-shot learning to predict the class of disease. As a result of this few-shot learning approach, less training data may be needed for disease classification. A greater number of classifiers trained to classify a greater number of diseases may be created. The machine-trained classifier(s) is applied to medical data of a patient to diagnose that patient and/or for clinical decision support.

In a first aspect, a method is provided for disease classification in a medical system. A medical scan of a patient is acquired. The disease of the patient is classified from the medical scan. The classifying uses input of data from the medical scan to a first machine-learned model having been trained for classification with few-shot learning from a second machine-learned model having been trained for prediction of functional or anatomical characteristics. A classification from output by the first machine-learned model in the classifying is displayed.

In one embodiment, magnetic resonance scan data is acquired. Cardiac disease is classified with the first machine-learned model where the second machine-learned model was trained for prediction of ejection fraction.

Various models may be used for the initial model to predict function or anatomy characteristic. For example, a multi-task model is used. In one embodiment, the initial model (second model) and the classifier (first model) are neural networks.

Various types of few-shot learning may have been performed. In one embodiment, the first machine-learned model was trained with few shot learning where the training used episodes and a long-short term memory. The few-shot learning allows for a fewer number of samples of training data, such as less than 200 samples. The initial model (e.g., second model) may have been trained with many more samples, such as at least 1,000 samples. There may be very few (e.g., less than 100) samples from actual patients for the few shot learning. The number of samples may be increased by generating synthetic examples. Similarly, the number of samples for training the initial model may be increased by generating synthetic examples. For example, the at least 1,000 samples include a first set of samples from people and a second set of samples including the synthetic examples. For dealing with outliers, the numbers of values of ground truth provided by the first set of samples has a first variance, and the number of values of the ground truth provided by the second set of samples reduces the first variance.

To account for missing and/or noisy labels, the second machine-learned model may have been trained with weak supervision as a labeling function. In one embodiment, an uncertainty of the classification is estimated. The uncertainty is output with the classification.

The classification may be used for clinical decision support. A processor generates a clinical decision from the classification. The machine-learned model may have been trained to output the clinical decision with or instead of the classification. An uncertainty of the classification may be estimated and used to generate the clinical decision based on the uncertainty.

In a second aspect, a method is provided for machine training for disease classification. An anatomical or functional characteristic linked to a pathology is identified. Data samples of patient data having known values of the anatomical and/or functional characteristics are located. A first classifier is machine trained with the data samples as training data where the known values are ground truth. The first classifier is machine trained to output the anatomical or functional characteristic. A second classifier adapted from the machine-trained first classifier is machine trained. The second classifier is machine trained with few-shot learning to output the pathology. The machine-trained second classifier is stored for later application to new patients.

In one embodiment, the anatomical or functional characteristic is identified as ejection fraction, and the pathology is a type of cardiac disease. In other embodiments, some of the training data is generated as synthetic samples derived from the data samples. Various differences in number of examples in training data may be used. For example, the machine training of the first classifier uses training data with a number of examples at least ten times a number of examples for machine training the second classifier.

In one embodiment, the second classifier is machine trained where the few-shot learning uses data separation into episodes. In another embodiment, an uncertainty of the output of the second classifier is predicted based on the machine training of the second classifier.

In a third aspect, a medical imaging system is provided for cardiac classification. A medical imager is configured to scan a patient. An image processor is configured to classify a cardiac condition of the patient from output of a few-shot machine-trained model adapted from a multi-task trained initial model where multiple tasks of the multi-task trained initial model are anatomical and/or functional characteristics linked to the cardiac condition. A display configured to display information derived from the cardiac condition.

These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one embodiment of a method for machine training for disease classification;

FIG. 2 is an example graph showing distribution of known values of EF;

FIG. 3 shows example end-systole and end-diastole masks corresponding to a short axis slice of a patient;

FIG. 4 shows example original and synthetic end-systole and end-diastole masks corresponding to a short axis slice of a patient, where the synthetic masks are generated by interpolation;

FIG. 5 shows example original and synthetic end-systole and end-diastole masks corresponding to a short axis slice of a patient, where the synthetic masks are generated by affine transformation;

FIG. 6 shows example masks and corresponding example generated images from the masks for forming synthetic images;

FIG. 7 shows one embodiment of a machine-learned generator for generating synthetic masks;

FIG. 8 shows another embodiment of a machine-learned generator for generating synthetic masks;

FIG. 9 is an example graph of FIG. 2 showing distribution of known values of EF after padding by synthetic samples;

FIG. 10 is a flow chart diagram of one embodiment of a method for disease classification in a medical system; and

FIG. 11 is a block diagram of one embodiment of a medical imaging system for cardiac classification.

### DETAILED DESCRIPTION OF EMBODIMENTS

Few-shot learning is applied to disease classification. For example, few-shot learning is applied to magnetic resonance (MR)-based cardiac function assessment and disease classification in a multi-task framework. The few-shot learning (classification) leverages the knowledge encapsulated in anatomical and/or functional characteristics and is able to generate an efficient disease classifier using few data samples. In one embodiment, the workflow to generate the model using few-shot learning for classification is based on medical data, including imaging and non-imaging, deep learning then few-shot learning, multi-task learning, and/or uncertainty quantification.

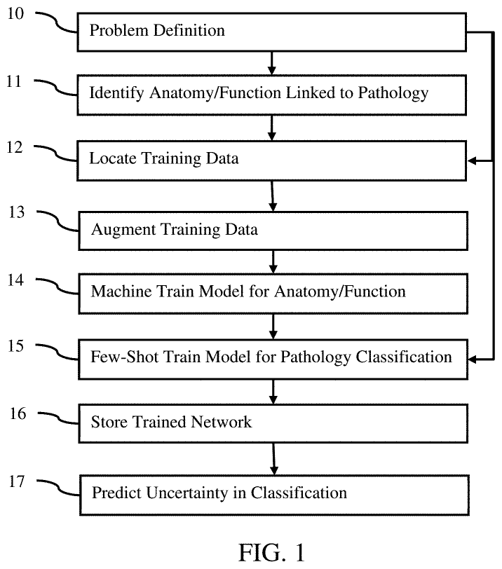

FIG. 1 shows a flow chart of one embodiment of a method for machine training for disease classification. Few-shot learning is to be used to create a machine-learned classifier (e.g., model or network) from a low number (e.g., 300, 200, or 100 or fewer) of training samples for the disease. Regional, hospital, medical practice, country, or type of patient specific classifiers may be trained for any of many different diseases given the ability to train with a low number of training samples for the disease classification. The few-shot learning uses training for a functional and/or anatomical characteristic with many samples in order to then learn disease classification with a few samples.

The method is implemented by a machine (e.g., computer, processor, workstation, or server) using training data (e.g., samples and ground truths for the samples) in a memory. Additional, different, or fewer acts may be provided. For example, acts 10, 11, 12, 13, and/or 17 are not provided. As another example, acts for repeating the method for other medical groups and/or diseases are provided.

In act 10, the problem is defined. A pathology and corresponding desired classification (e.g., type or subtype of cardiac disease as the pathology) are established. For example, cardiomyopathy is the pathology, so a classifier is to be trained to determine whether a patient has cardiomyopathy and/or to determine a level of cardiomyopathy. Other example cardiovascular pathologies include coronary artery disease, myocardial infarction, heart valve disease, or systolic heart failure. Other types of diseases and/or subtypes may be selected.

The type of input data available for the pathology is identified in order to gather training data for classification as well as the initial modeling of a linked functional and/or anatomical characteristics. By identifying the available data, the types of information that may be used for classification and/or training are established.

In act 11, an anatomical or functional characteristic linked to the pathology is identified. An expert, such as a physician, may identify. In other embodiments, a processor identifies. For example, natural language processing (NLP) performs an automated search in the medical literature to identify the list of anatomical and/or functional characteristics linked with the pathology. The characteristics linked to the types and subtypes of the pathology are identified. The search may further limit the characteristics to ones in or identifiable in the type of input data available for training and/or in clinical practice.

Examples of anatomical characteristics include: linear measurements such as linear internal measurements of the left ventricle and its walls; volumetric measurements such as time-varying volume, end-systolic volume, and/or end-diastolic volume; left ventricle (LV) mass; size of the cardiac valves opening and size of the valve leaflets; quantification of abnormal valve anatomy (e.g. bicuspid aortic valve); and/or quantification of stenosis (e.g. in the aorta). Other anatomical characteristics related to size, shape, mass, abnormality, and/or restriction may be used.

Examples of functional characteristics include: ejection fraction, stroke volume, regional wall motion (e.g., normal=1, hypokinesis=2, akinesis=3, dyskinesis=4), myocardial perfusion at rest and/or hyperemia, quantification of valve regurgitation, and/or presence of delayed enhancement (LGE). Other functional characteristics related to operation or performance of anatomy may be used.

Other types of characteristics may be used, such as quantitative tissue parameters (e.g., T1, extracellular volume (ECV), T2, T2*, BOLD, and others). For any given pathology, different characteristics may be associated with the pathology. The characteristics linked (causal and/or correlational) to the pathology and available in medical data are identified. All or a sub-set (e.g., one) of the identified characteristics are then used for initial machine training of a model to be adapted for classification using few-shot learning.

In one embodiment, the pathology is a type of cardiac disease, such as cardiomyopathy. The ejection fraction is identified as the anatomical or functional characteristic linked to the pathology.

In act 12, data samples of patient data having known values of the anatomical and/or functional characteristics are located. A processor searches patient medical records to locate samples having known values or determinable values of the characteristic(s). Past datasets that match the type of input data and the anatomical and/or functional characteristics identified in act 11 are found.

The located data samples may be reformatted, such as extracting the values of the characteristics and any medical data of interest and arranging the data in a spreadsheet or table as training data. The medical data of interest may the MRI scans (e.g., scan data), images from the MRI scanning (e.g., scan data), other imaging data, and/or non-imaging data (e.g., clinical tests, patient notes, medical history, . . . ). The data to be used as input for classification is located and formatted for machine training.

For example, cardiac MRI scans may be available for hundreds or thousands of past patients. The cardiac MRI datasets are identified for which the EF ground truth value is either known or can be derived (e.g. by visual assessment, manual segmentation, etc.). Heart chamber volume or stroke volume are alternative or additional parameters for the ground truth. Other parameters may be used. The cardiac MRI scan data with or without other medical data (e.g., family history, blood pressure, ECG, . . . ) is located for each sample and formatted as training data.

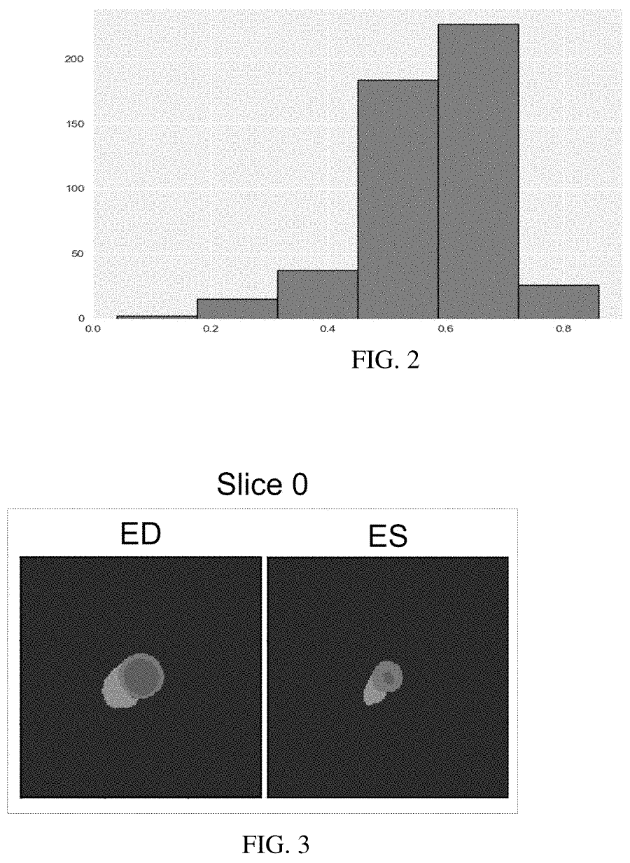

Typically, the identified datasets would have more values of the characteristics in a healthy range assuming more patients do not have the particular disease of interest. FIG. 2 shows an example distribution of EF values by percentage (shown as a 0.0-1.0 on the x-axis) with the number of past patients having the EF values. The EF values are mostly in the healthy range (50-70%). The search may be continued to locate more datasets in the unhealthy range. Additionally or alternatively, the located data is augmented with synthetically created data to increase the number of training data samples and/or to reduce variance of numbers of samples for different ground truths.

In act 13 of FIG. 1, a processor generates additional training data as synthetic samples derived from the data samples of actual patients and/or simulation. Some of the training data will be actual data and some of the training data will be synthetically created samples. In this optional act 13, the dataset to be used as training data is augmented with synthetic datasets.

The distribution of the values of the anatomical and/or functional characteristics may not be uniform (see FIG. 2). Hence, to be able to train an accurate model for the anatomical and/or functional characteristic, data augmentation based on synthetic data may be used to generate a more uniform distribution in the dataset. Overall, the use of synthetic data during the training phase may provide several advantages. A very large number of cases can be automatically generated, leading to an extensive database including rare cases and/or complex configurations in the sampling. For example, different combinations of anatomical and/or functional characteristics not frequently in actual patients are created. The augmentation may be done in a way to ensure that a wide range of values for the anatomical and/or functional characteristics is present in the augmented dataset. The classifier is to be trained for the entire range of values to be more accurate, so a larger number of datasets at the two extremes are created.

The synthesis is used to create training data for training the initial model to predict the characteristic and/or for training the later model adapted from the initial model to predict the disease classification. The generation of synthetic data with specific anatomical and/or functional characteristics is easier to perform than the generation of synthetic data corresponding to a certain disease type and/or subtype. Reliable disease classification may be more accurate by experienced clinical personnel. The assessment of anatomical and/or functional characteristics on medical data can be performed by non-clinical personnel or in an automated manner. Since the generation of synthetic data can be automated, the cost of generating a large database is reduced.

The synthetic data is data from the actual patients altered to provide a new value of the characteristic. Alternatively, the synthetic data is generated by simulation, such as by altering a patient model or a statistical model of a patient population and then simulating imaging and/or other data gathering from the patient model. In yet other approaches, the synthetic data is generated by altering a physical model of part of the patient and measuring the characteristics from the physical model. The synthetic data does not represent any actual patient collected for the training data but is instead synthesized.

In one embodiment where datasets for EF are synthesized based on a statistical model, a generative adversarial network (GAN), such as GauGAN, or another generator (e.g., image-to-image or U-Net neural network) creates scan data from input masks. The masks are synthesized for different EF to then create the scan data (e.g., cardiac MRI). For example, the GauGAN model may be trained for the generation of synthetic images using pairs of masks and corresponding real images from patients. Once the GauGAN model is trained, synthetic masks are generated (e.g. for ED and ES), which the GauGAN model can then transform into synthetic images as samples of input data with the ground truth EF being based on the volumes represented by the ED and ES masks.

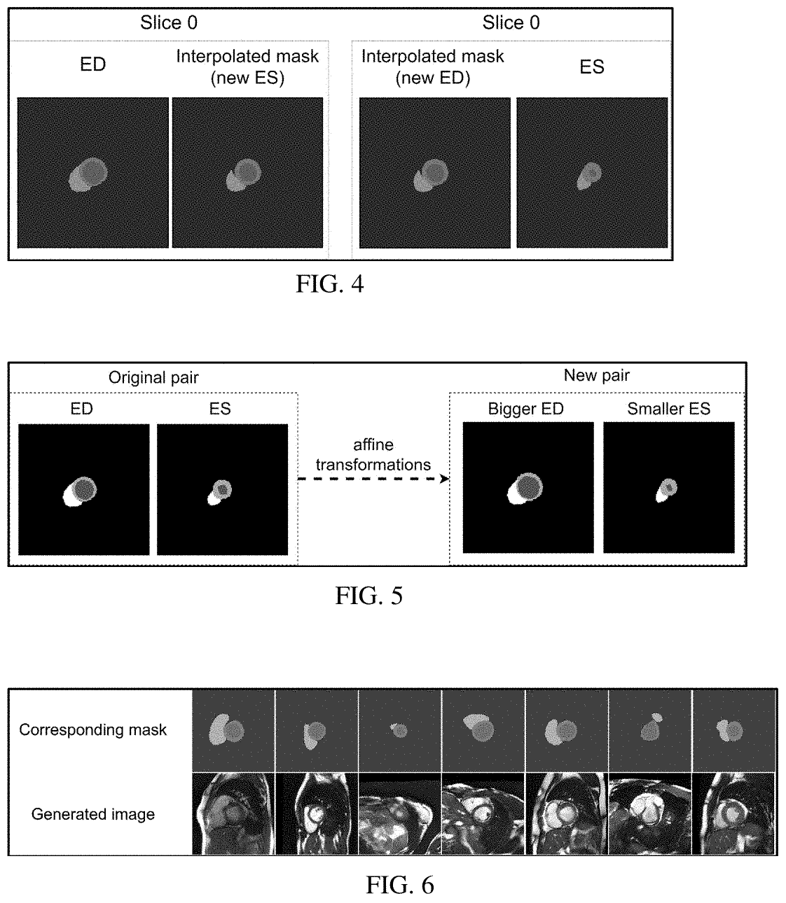

Actual patient data or simulated masks may be used as the starting point. For example, the masks at end-diastole (ED) and end-systole (ES) from actual patient datasets are used as seeds. FIG. 3 shows example ES and ED masks for the first SAX slice of a patient. In one embodiment, the synthetic masks are generated by interpolation from the actual masks. For example, the interpolated mask is generated as: Interpolated mask=(α*SDT₁)+((1−α)*SDT₂) where SDT₁ and SDT₂ are signed distance transform masks of ED and ES of the actual patient, and a is a parameter provided as input, which may take values between 0 and 1. Thus, new pairs of ED and ES are formed: (ED, interpolated mask) and (interpolated mask, ES). FIG. 4 shows an example of synthesized new pairs where the actual mask is used for part of each pair. In FIG. 4, one pair is (ED, interpolated mask as new ES) and (interpolated mask as new ED, ES). Both synthesized pairs belong to slice 0 of two new different synthetic patients.

Where the objective is to generate datasets with a reduced EF value, affine transformations are employed to rescale the ED and ES masks. The affine transformation may be applied to the original or actual patient masks and/or to synthesized masks. The affine transformation makes the anatomical structures smaller at ES and/or larger at ED. These synthesized (ED, ES) pairs are used to create new synthetic patients with larger EF. FIG. 5 shows an example using affine transformation to generate new (ED, ES) pairs where ED is larger, and ES is smaller. This transformation may be applied to every slice in order to obtain synthetic patients with higher EF values.

The opposite affine transformation may be used to generate datasets with larger EF values. Any or different affine transformations may be used. By using different transformations, additional synthetic sample mask pairs may be generated. Rigid transformation may be used.

The affine transformation may be used to generate synthetic examples for any desired EF values. By controlling the transformation, the number of synthetic examples at the different EF values may be established. To control the number of these newly generated pairs, and implicitly the EF values, a parameter γ is provided as input. The parameter is a scale or weight for the increase and/or decrease in size. Multiple uniformly distributed sample values of γ over the interval [1-N, 1) are used for rescaling the ES mask, leading to a smaller LV for ES and implicitly a smaller volume. Non-uniform distribution may be used. The same number of samples is used for the ED mask, but over the interval [1, 1+N), resulting in a larger LV for ED and an increased EF for the patient. N is also a parameter provided as input, which may be set between 0.5 and 0.2, and which controls the contrast between the ED LV size and the ES LV size. By controlling α, γ, and N, the transformation is controlled to provide the desired synthesized samples. In this approach, the synthetic datasets are generated starting from real patients. The number of new patients is controlled by parameters α and γ.

FIG. 6 shows some example synthetically generated masks. Using GauGAN, corresponding MRI slice images are generated as shown in FIG. 6. The EF values are known for the synthetically generated samples from the end-systole and end-diastole volumes represented by the masks at a given time over the range of slices. The generated synthetic images are generated to be used as inputs to the model to be trained to estimate the EF.

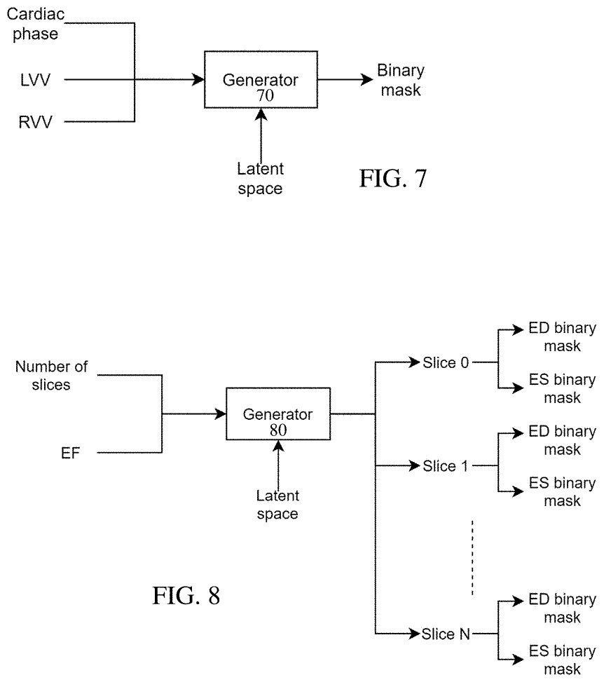

In another embodiment, a neural network is used to generate synthetic samples as training data (i.e., input data such as medical images (scan data) and output data such as EF). FIG. 7 shows one approach with a deep neural network, called a generator 70, produces individual frames of masks. The mask may be binary, such as binary for each of multiple different structures, or may be a mask with three or more levels to distinguish three or more structures. The inputs of this generator 70 are cardiac phase to handle differences between ED and ES (e.g., myocardium thickness), the volume of the left ventricle (LVV), and the volume of the right ventricle (RVV). The generator 70 is trained to generate masks given different input values. The resulting EF and each individually generated frame may be accurately controlled by variation of the input values. The EF is known from the LVV and RVV at the end-diastole and end-systole cardiac phases. The generator 70 is used to generate masks for different slices or separately slice-specific generators 70 are used. By sequential application, the generator(s) 70 generate ED, ES mask pairs for the different slices. An extra layer of generation may be added to aggregate all the generated individual frames into a synthetic patient dataset, such as providing ED and ES binary masks. A latent space consisting of an n-dimensional hypersphere vector may be provided as input to obtain a larger variability. This vector can be further used to control the style of the frames.

FIG. 8 shows another approach with a deep neural network as a generator 80. A synthetic patient dataset (e.g., both ED and ES for all slices) is directly produced. The input of the generator 80 is the desired number of slices, N, and the desired EF value or desired volumes for ED and ES. The generator 80 then generates two binary masks (ED and ES) for every slice. The latent space is also provided as input to provide variability in the resulting images. The generator 80 directly produces synthetic patients with specific EF values. Other parameters can be provided as input like slice gap, slice thickness, resolution, etc.

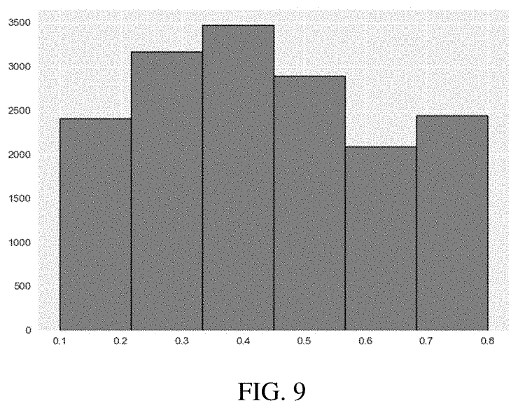

The synthetic examples are combined with the actual patient examples. Using the controls for generating the synthetic examples, a quasi-uniform EF distribution may be obtained. FIG. 9 shows the graph of FIG. 2 with the synthetic examples being added. Many samples are generated with more samples in the unhealthy ranges so that the variance of FIG. 2 is reduced as shown in FIG. 9.

In act 14 of FIG. 1, a processor machine trains a classifier. The classifier is a machine learning model to be trained to estimate the values of one or more functional and/or anatomical characteristics. The initial classifier is formulated as a single- or multi-task problem to perform a classification of the data samples in the dataset based on the anatomical and/or functional characteristics.

In act 15, the trained initial classifier is adapted for few-shot learning to learn with much less training data to estimate the disease (e.g., level of cardiomyopathy). The end goal is a machine-learned model to classify for the disease. The knowledge encapsulated in a model trained for characteristic (e.g., EF) prediction will be leveraged to obtain a machine-learned model for disease diagnosis and classification with few training data samples (with annotated diseases diagnosis and classification). Since there is a strong link between the characteristic (e.g., EF) and multiple pathologies, the initially trained model to predict the characteristic may result in accurate prediction of the disease class using few-shot learning in act 15.

The initial classifier is trained with the data samples as training data. The data samples are from actual past patients having a known ground truth, such as known values of EF. Alternatively, the ground truth is derived from the data samples and then used in training. The data samples may also include augmented examples. For example, many samples both actual and synthesized having input data (e.g., medical data) and known values for one or more characteristics to be predicted by the classifier are used as training data. The medical data is used as input, and the known values are used as ground truth. The classifier is to be machine trained using the training data to output the anatomical and/or functional characteristic, such as inputting medical data including scan data and outputting a value for EF. The training data for the initial classifier (model) has many samples, such as thousands or tens of thousands. The training data for the few-shot learning to be performed using the first or initial classifier as a starting point in act 15 has fewer training data samples, such as at least ten times fewer (e.g., tens or hundreds).

The output may have any resolution. For example, the EF is output as being in one of several ranges (e.g., seven ranges as multiple bins: <30%, 30-40%, 40-50%, 50-60%, 60-70%, >70%). Binary or other numbers of ranges may be used. A continuous output may be used.

Any machine-learning network or classifier may form the model. For learning features as part of the training (i.e., deep learning), the model is a neural network. Other networks or classifiers may be used. The classifier is a neural network but other machine training may be used.

The classifier includes outputs for one or more tasks. For example, an autoencoder, image-to-image network, U-net, fully-connected neural network, or convolutional neural network is used to output an estimation for one task, such as output values of EF. To provide a more accurate classifier of the disease, the initial classifier may be trained as a multi-task classifier where more than one characteristic linked to the pathology is predicted. For example, a neural network is defined with an architecture having two or more outputs. A multi-task classifier is used so that the classifier is trained to optimize for the multiple tasks, such as including a loss function based on multiple losses (i.e., a loss for each of the tasks). By forcing the network to also learn other related tasks, potentially the performance obtained on the main tasks of interest increases.

In one example, the multi-tasking network is defined to perform heart-chamber segmentation as one task, and EF classification or regression (separate from the segmentation) as another parallel task. Classification (e.g., using visual assessment as ground truth) and/or regression (e.g., using segmentation as the ground truth) may be used in learning to estimate the EF value. For example, the same network performs the segmentation and EF estimation. This way the network is ‘forced’ to learn both based on annotations and on (e.g. visual) clinical assessments. The level of agreement between the two or more tasks of quantifying cardiac function can be used to measure the certainty of the EF prediction. For example, the result of EF classification and quantitative EF derived from the segmentation result could be combined into an ensemble model. In another example, training the two parallel tasks (segmentation and EF classification) in parallel in the same network may lead to a more robust EF quantification. If training data were available with no segmentation ground truth, it could still be used for training one of the tasks, so the latent space encoding of the multi-task network would learn the representation of those images without a segmentation ground truth.

For machine learning with a neural network, the architecture of the model is defined. The architecture may have any number and/or type of layers, nodes, activation functions, learnable parameters, or other structures. In one embodiment, the architecture is defined as a multi-task architecture. The network architecture includes an output layer for each task, such as one output layer for segmentation or estimation of image features (e.g., handcrafted radiomic features) and another output layer for EF. Any generative architecture may be used for unsupervised learning to predict segmentation, EF, and/or other anatomical or functional characteristics. For example, a convolutional neural network or a fully connected neural network is provided.

The definition is by configuration or programming of the learning. The number of layers or units, type of learning, order of layers, connections, and other characteristics of the network are controlled by the programmer or user. In other embodiments, one or more aspects of the architecture (e.g., number of nodes, number of layers or units, or connections) are defined and selected by the machine during the learning.

The defined model (e.g., neural network) is trained to generate outputs for one or more tasks, such as multiple tasks. The model is trained by machine learning. Based on the architecture, the model is trained to generate output using the training data to find optimum values for learnable parameters of the model.

The training data includes many samples (e.g., hundreds or thousands) of input medical data (e.g., scan data with or without non-imaging data) and ground truths (e.g., EF or EF and segmentations). The ground truths may be annotations from experts or data mined from patient records, such as outcomes or segmentations for the samples. The ground truths may be automatically determined from the input, such as segmentation or radiomic features. The network is trained to output based on the assigned ground truths for the input samples.

For training, various optimizers may be used, such as Adadelta, SGD, RMSprop, or Adam. The weights of the initial model are randomly initialized, but another initialization may be used. End-to-end training is performed, but one or more features may be set. The network for one task may be initially trained alone, and then used for further training of that network for the one task and a further network for the other task. Separate losses may be provided for each task. Joint training may be used. Any multi-task training may be performed. Batch normalization, dropout, and/or data augmentation are not used, but may be (e.g., using batch normalization and dropout). During the optimization, the different distinguishing features are learned. The features providing an indication of outcome and indication of another task are learned.

The optimizer minimizes an error or loss, such as the Mean Squared Error (MSE), Huber loss, L1 loss, or L2 loss. The same or different loss may be used for each task. In one embodiment, the machine training uses a combination of losses from the different tasks.

Data programming and/or noisy labels may be used to allow the model to learn from as many data and ground truth sources as possible. For example, for the same functional or anatomical characteristic (e.g. ventricular diameter), each training data sample may include an image (scan data) and multiple ground truth values (e.g., ground truth labels from a measurement performed by the clinician in the clinical setting, visual estimation, and a value extracted from an annotated contour). Although the different sources refer to the same characteristic of the same patient, in general there will be differences between these values. The values for the same characteristic for the same patient may even conflict. Data programming and/or noisy labels allow the model to learn to extract information from all labels and offer a superior and more robust performance.

Noisy training labels may be exploited by specifically encoding a weak supervision in the form of a labeling function. Labeling functions may have widely varying error rates and may conflict on certain data points. The labeling functions may be modeled as a generative process, leading to an automated denoising by learning the accuracies of the labeling functions along with their correlation structure. A labeling function need not have perfect accuracy or recall; rather, it represents a pattern that the user wishes to impart to their model and that is easier to encode as a labeling function than as a set of hand-labeled examples. The labeling function can be based on external knowledge bases, libraries or ontologies, can express heuristic patterns, or some hybrid of these types. The use of a labeling function is more general than manual annotations, as a manual annotation can always be directly encoded by a labeling function. The labeling function may overlap, conflict, and even have dependencies which users can provide as part of the data programming specification. Some of the values of the labeling functions may not be available for all patients. Another advantage is that missing label values are also allowed (e.g. the visual assessment may be missing in some cases). For each labeling function, an ‘abstain’ value may be assigned.

Once trained, the initial machine-learned model outputs a value or values of functional and/or anatomical characteristics in response to input of previously unseen medical data. The goal is to alternatively or additionally output a classification of the disease of interest, such as cardiomyopathy, linked to the characteristics.

In act 15, the initial classifier is adapted to machine train another classifier. The other or subsequent classifier is trained to output a classification of disease. There is less training data available for disease class, so the subsequent classifier is machine trained with few-shot learning to output the pathology. The training dataset for disease class may be very small, such as 200 or fewer samples. The number of types and subtypes of CVD is very large making locating a large number of samples for one type or subtype difficult, costly, and/or time consuming. Moreover, disease classification is continuously changing and can vary largely from region to region. Few-shot learning is used to allow training from a few samples more easily available in the variable environment. Few-shot learning may be used by a clinical center to develop an algorithm for a novel classification that is not yet part of established guidelines.

The machine training may use the same or different optimization, loss function, learnable parameters, and/or other structure as the machine training for the initial classifier. The initial classifier provides values for the learnable parameters as a starting point or initialization for the few-shot learning. Since the initial classifier for the linked characteristic(s) is the starting point, the architecture is adapted for disease classification. A classification output layer or layers are added. For example, one or more fully connected layers and a SoftMax layer are added to receive the estimate of EF and output the classification. As another example, one or more layers are added in parallel to the output of EF so that one or more intermediate layers output feature values to both the layers for estimation of the characteristic and to layers for the estimation of the disease classification. In training, all or only a sub-set of the previously learned parameters of the classifier are altered. Some learned parameters may be fixed for the few-shot learning. The characteristic output is not used with a loss function, instead machine training only for the disease classification. Alternatively, the few shot learning is trained as a multi-task network where the characteristic(s) and disease classification losses are used together to optimize the learnable parameters.

The machine learning fine-tunes the initial classifier as adapted for disease classification to diagnose one or more pathologies based on only very few training samples. For example, using the few-shot learning approach, the model is initially trained on a large database to distinguish between different EF bins, and then, given 50-100 datasets of patients with different levels of cardiomyopathy, the adapted model is fine-tuned to distinguish between severe and non-severe cardiomyopathy. Few-shot learning reduces the overfitting compared to the case where a network would be trained directly or initially on the 50-100 datasets.

The output layer(s) and corresponding learnable parameters added for disease classification may be for binary and/or continuous output. In various embodiments, the classifier is fine-tuned from the initial classifier to perform binary classification of a single pathology, N-ary classification of a single pathology, binary classification of each of multiple pathologies, N-ary classification of each of the multiple pathologies, or binary for one or more pathologies and N-ary for one or more other pathologies.

The few-shot learning technique leverages a small training dataset and the initially trained classifier to obtain a model for disease diagnosis and classification. In few-shot learning, the initial machine learning model is adapted to accommodate new classes not seen during training, given only a few examples of each of these classes, or to accommodate a new task for which only little annotated data is available. A naive approach, such as re-training the model on the new data, would severely overfit. One few-shot learning strategy is to synthetically augment the small amount of data available for the new class or new task. Another few-shot learning strategy is to design a multi-task network trained to perform at least two tasks: one task for which training data is abundant, for e.g. cardiac segmentation, and a second related task for which data is scarce, e.g. detect subjects with a rare condition such as left-ventricular non-compaction or left-ventricular obstruction. In this example, sharing network parameters between the two tasks improves the robustness and generalizability of the second task for which little data is available. Another few-shot learning strategy is to use an embedding, where data is compressed into another representation where similar samples are grouped together without using knowledge about the final classes that are desired. Then the classification task is trained on the smaller embedded space, which reduces the number of training samples needed. Few-shot learning separates the limited number of training data samples into sub-sets for training. For example, the training data is separated into episodes to maximize the optimization of the network through a sequence of episodic machine training.

In one embodiment, matching networks use an attention mechanism over a learned embedding of the labeled set of examples (the support set) to predict classes for the unlabeled points (the query set). Matching networks can be interpreted as a weighted nearest-neighbor classifier applied within an embedding space. This model utilizes sampled mini-batches called episodes during training, where each episode is designed to mimic the few-shot task by subsampling classes as well as data points. The use of episodes makes the training problem more faithful to the test environment and thereby improves generalization. In another embodiment, a meta-learning approach to few-shot learning is used. A long-short-term memory network (LSTM) structure or layers are added to produce the updates to a classifier, given an episode, such that the LSTM will generalize well to a test-set. Rather than training a single model over multiple episodes, the LSTM meta-learner learns to train a custom model for each episode. The outputs of the episodic models are combined to provide a class output. In yet another embodiment, the classifier should have a simple inductive bias to avoid overfitting due to the few number of training samples. Prototypical networks based on the idea that there exists an embedding in which points cluster around a single prototype representation for each class are used. In order to do this, a non-linear mapping of the input into an embedding space is trained using a neural network, and a class's prototype is taken to be the mean of its support set in the embedding space. Classification is then performed for an embedded query point by finding the nearest class prototype. Other few-shot learning approaches may be used.

In act 16 of FIG. 1, the machine-trained classifier is stored. The disease classifier resulting from the few-shot learning is stored. The model parameters, such as connections, convolution kernels, weights, or other learned values for the network, are stored. The network is stored in memory to be used for application or testing.

Once trained, the classifier may be applied to classify whether or not a disease state exists and/or the level of disease of a patient. The many samples in the training data are used to learn to output given an unseen sample, such as a scan volume from a patient. The trained classifier outputs a disease classification in response to input of the medical data for a patient. The machine-learned classifier may be used to classify for any number of patients.

In act 17, the processor predicts an uncertainty of the output of the machine-trained disease classifier based on the machine training of the disease classifier. The confidence of the disease classification is quantified. Reliable assessment of the confidence of automated disease diagnosis and classification may be of interest to physicians or other processes relying on the classification. In one embodiment, following the approach of the prototypical networks, a function assessing the confidence of the prediction may be defined based on one or more sources of information extracted during training and inference. For example, in training, the distance between clusters and/or class prototypes during the initial training of the network (focusing on anatomical and/or functional characteristics) is determined. The larger the distance (i.e., the better the clusters and/or class prototypes are separated from each other), the higher will the confidence be. As another example in training, the distance between clusters and/or class prototypes focusing on disease diagnosis and/or classification is calculated. In an example in inference, the distance between the current sample and the clusters and/or class prototypes is evaluated to determine the clusters and/or class to which the current sample pertains. The lower the distance between the current sample and a cluster and/or class prototype is, the higher is the confidence of the prediction. Other sources of confidence information may be provided. The final confidence metric may be defined as a function of these distances, where different weights may be associated to each one them. In other embodiments, uncertainty based on probability distribution of the ground truth and/or operation of the trained classifier relative to the ground truth (functional or anatomical characteristic and/or disease classification) is calculated and output.

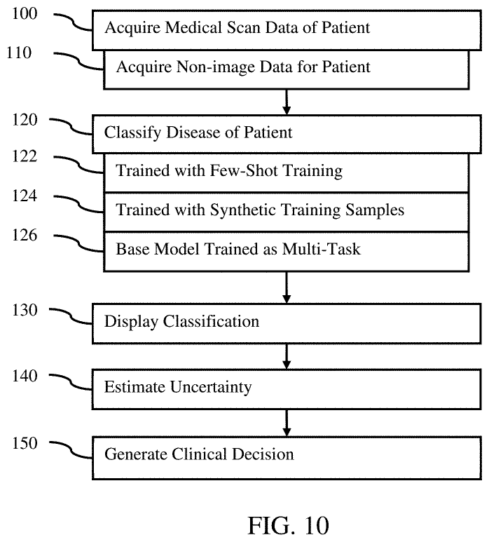

FIG. 10 is a flow chart diagram of one embodiment of a method for disease classification in a medical system. A few-shot machine-learned classifier or model is used to classify disease state of a patient based on input for that patient. The stored classifier is applied, such as estimating EF and classifying a level of cardiomyopathy for the patient based on input of scan data with or without other medical data to the machine-learned classifier. Different classifiers may be used for different pathologies or one classifier is used for one or multiple pathologies.

The method is performed in the order shown (e.g., top to bottom or numerical), but other orders may be used. For example, acts 130 is performed after act 140 or act 150. As another example, acts 140 and 150 are performed in opposite order.

Additional, different or fewer acts may be provided. For example, acts 140 and/or 150 are not performed. As another example, acts 110, 124, and/or 126 are not performed.

The method is performed by a medical diagnostic scanner, a workstation, a server, or a computer. The scanner or memory are used to acquire data for a patient. An image processor, such as an image processor of the scanner or a separate computer, applies the machine-learned model and classifies disease state. The image processor displays using a display screen or printer. A physician may use the output information to make a treatment decision for the patient.

In act 100, an image processor acquires a medical scan of a patient. The scan data from the scan of the patient is acquired from a medical scanner, such as a computed tomography or MR scanner. The computed tomography scanner scans a patient with x-rays using an x-ray source and detector mounted to a gantry on opposite sides of a patient. A magnetic resonance scanner scans a patient using pulses in a magnetic field and detecting energy due to spin re-orientation of molecules in the patient. A positron emission tomography, single photon emission computed tomography, or ultrasound scanner may be used. In alternative embodiments, scan data from a previous scan of the patient is acquired from a memory or transfer over a computer network.

The input of the medical system is a medical image, such as scan data. The scan data represents an area or volume of the patient. For example, the scan data represents a three-dimensional distribution of locations or voxels in a volume of the patient. The distribution of locations may be in a Cartesian coordinate system or uniform grid. Alternatively, an non-uniform grid or polar coordinate format is used. For representing a volume, a scalar value is provided for each voxel representing the volume.

The scan data may be pre-processed before application to machine-learned classifier. Pre-processing may include segmentation, filtering, normalization, scaling, or another image processing. For example, one or more tumor volumes (e.g., gross tumor volume) or regions including the tumor with or without non-tumor tissue are segmented. The segmentation may be by manual delineation or automatically by the image processor. The scan data to be input represents just the segmented region or separate inputs are provided for the segmented region and the entire scan volume.

The pre-processed scan data (e.g., image data) is used alone to predict outcome. Alternatively, both the pre-processed scan data and scan data with more or less processing are input to predict outcome. Non-image data may be input instead or in addition to scan data.

In act 110, the image processor acquires non-image data. The non-image data is from sensors, the computerized patient medical record, manual input, pathology database, laboratory database, and/or other source. The non-image data represents one or more characteristics of the patient, such as family history, medications taken, temperature, body-mass index, and/or other information. For example, genomic, clinical, measurement, molecular, and/or family history data of the patient are acquired from memory, transform, data mining, and/or manual input.

In act 120, the image processor classifies the disease of the patient from the medical data for the patient. The classification uses artificial intelligence. The classification is based on input of the scan data (e.g., voxel data) and/or non-image data to the few-shot trained model. For example, voxel data for a segmented three-dimensional region of the heart and/or circulatory system and surrounding tissue is input, and the output is a level of cardiomyopathy. The few-shot machine-learned classifier or model classifies based on the input.

Act 120 is represented in FIG. 10 as having three components, classification based on the model having been trained with few-shot learning in act 122, the model having been trained using, at least in part, synthetic training samples in act 124, the model having been trained, in part, as a multi-task model in act 126. One, any two, all three, or none of these components may be used in various embodiments. In one embodiment, acts 124 and 126 are not used.

In act 122, the image processor classifies using a machine-learned model that was trained for classification with few-shot learning from another machine-learned model having been trained for prediction of functional or anatomical characteristics. The machine-learned model for disease classification was trained with the few shot learning where the training used episodes, a long-short term memory, classifiers in different stages, and/or prototypical networks.

The type of training, training data, and architecture of the machine-learned model affect the output classification. Differences in any of these training-related approaches may result in differences in the output classification. By having performed training in a certain way in the past, the machine-learned model performed differently in application.

The machine-learned model for disease classification and/or the machine-learned model for functional and/or anatomical characteristic estimation on which the model for disease classification is based may be any of various types of network. In one embodiment, the models are neural networks, such as convolutional or fully connected neural networks.

The model for characteristic estimation may have been trained with many samples, such as with at least 1,000 samples. The model for disease classification may have used few-shot learning with fewer training samples, such as less than 200 training samples. The few-shot learning limits or avoids overfitting given this small number of training samples.

In act 124, some or all of the training samples may have been synthetic examples (e.g., training data not reflecting an actual patient). The synthetic training data may have been for the samples used in training for classification (e.g., some of the less than 200 samples) and/or for the samples used in training for anatomical or functional characteristic estimation (e.g., some of the at least 1,000 samples). Some of the training samples may have been from actual people or patients. The synthetic samples may have been controlled in creation to reduce a variance in the sampling of the ground truth based just on training data from actual patients, such as to provide more samples of extreme or under-sampled values of the characteristic.

The classification is for a disease of interest, such as a cardiac disease. By having used the few-shot training and/or synthetic training data, a classifier may have been created for diseases with a few number examples. The initial training was for a characteristic linked to the disease, such as EF, for which a greater number of samples may have been available.

In act 126, the model for estimating the characteristic on which the disease classification is built was a multi-task model. Different tasks and corresponding loss functions were used to train the model. The tasks are linked to the disease, such as being segmentation for a disease region and EF. Multiple outputs may be generated in response to the input. For application, less than all the trained network may be used, such as training as a multi-task generator but only using the parts of the multi-task network that output the disease classification. Alternatively, the entire network or the parts that output the estimates for the different tasks are used, such as outputting values for the multiple characteristics as well as the disease classification.

In one embodiment, the machine-learned model for disease classification and/or the model for characteristic estimation were trained with weak supervision using a labeling function.

In act 130, a display displays an image of the classification. The display is a visual output. The image processor generates an image. The image may be output to a display, into a patient medical record, and/or to a report.

The displayed classification is from the output of the machine-learned model. The output of the model may be directly displayed, such as the classification from the output is the output (e.g., a class or percentage output by the model). The classification may be derived from the output of the model, such as the output being a color-coding representing classification output by the model. More than one classification may be output.

The estimated characteristics (e.g., EF value) and/or input information may be output. Other information may be displayed with the classification. The classification may be presented with or on (e.g., overlay or annotation) an image of the patient.

In act 140, the processor estimates an uncertainty of the classification. The uncertainty for each possible classification may have been previously calculated. The corresponding value of the uncertainty for an output classification is looked up. Alternatively, the uncertainty is calculated for a given output at the time of the output of the classification for a patient.

The estimated uncertainty is output with the classification. The physician may make better choices or be more informed by indicating both the classification and the level of uncertainty on the display.

In act 150, the processor generates a clinical decision from the classification. The classification may be part of a workflow for patient diagnosis, prognosis, and/or treatment. The classification is used to decide upon a level for a next act (e.g., level of treatment), whether to perform a next act, and/or for selection of a branch in the workflow. The classification is used for decision support.

In one embodiment, the uncertainty of the classification is used in the decision support. The processor generates the decision based on the level of uncertainty. For a new patient, the classification and uncertainty are determined using the classification model. Where the confidence in classification is high, a fully automated decision is taken. The confidence of the decision is high, for example, where the dataset is confidently classified into a certain disease class, so automated decisions may be taken. Where the confidence in the classification is medium (e.g., the dataset is classified into a certain disease class, but the distance between the dataset and the closest cluster and/or class prototype is relatively large), a semi-automated decision is taken. A clinical expert briefly reviews the case and confirms and/or revises the final results or decision. Where the confidence in the classification is low (e.g., the dataset cannot be clearly classified into a certain disease class, the distances between the dataset and multiple clusters and/or class prototypes are comparable), a manual decision is used. The clinical expert has to review the case in detail to take the final decision.

In alternative embodiments, the machine-learned model is for treatment planning or imaging control. Instead of or in addition to disease classification, a treatment or treatment level may be output. The treatment output is based on few-shot learning from training data of treatments. The image control adapts the acquisition protocol (e.g., cardiac MR acquisition) to further investigate the suspected disease (e.g., CVD).

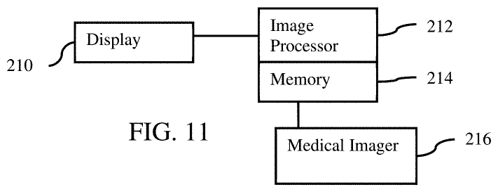

FIG. 11 shows a medical imaging system for cardiac classification. The system generates a classification on a display 210. The medical imaging system includes the display 210, memory 214, and image processor 212. The display 210, image processor 212, and memory 214 may be part of the medical imager 216, a computer, server, workstation, or other system for image processing medical images from a scan of a patient. A workstation or computer without the medical imager 216 may be used as the medical imaging system.

Additional, different, or fewer components may be provided. For example, a computer network is included for remote classification based on locally captured scan data. As another example, a user input device (e.g., keyboard, buttons, sliders, dials, trackball, mouse, or other device) is provided for user interaction with the classification.

The medical imager 216 is a computed tomography, magnetic resonance, ultrasound, positron emission tomography, or single photon emission computed tomography scanner. For example, the medical imager 136 is a MR system having coils or antennas and an electromagnet around a patient bed.

The medical imager 216 is configured by settings to scan a patient. The medical imager 216 is setup to perform a scan for the given clinical problem, such as a cardiac scan. The scan results in scan or image data that may be processed to generate an image of the interior of the patient on the display 210. The scan or image data may represent a three-dimensional distribution of locations (e.g., voxels) in a volume of the patient.

The image processor 212 is a control processor, general processor, digital signal processor, three-dimensional data processor, graphics processing unit, application specific integrated circuit, field programmable gate array, artificial intelligence processor or accelerator, digital circuit, analog circuit, combinations thereof, or other now known or later developed device for processing medical image data. The image processor 212 is a single device, a plurality of devices, or a network. For more than one device, parallel or sequential division of processing may be used. Different devices making up the image processor 212 may perform different functions. In one embodiment, the image processor 212 is a control processor or other processor of a medical diagnostic imaging system, such as the medical imager 216. In alternative embodiments, the image processor 212 is a processor for operating on non-image data. The image processor 212 operates pursuant to stored instructions, hardware, and/or firmware to perform various acts described herein.

In one embodiment, the image processor 212 is configured to train one or more machine learning networks. Based on a user provided or other source of the network architecture and training data, the image processor 212 learns values for learnable parameters of the network. A single or multi-task generator is trained using ground truth and corresponding losses for the functional or anatomical estimation tasks. The tasks are for characteristics linked to a pathology. The processor 212 then performs few-shot learning to fine tune the trained characteristic network for classification of disease.

Alternatively or additionally, the image processor 212 is configured to apply one or more machine-learned networks, models, or classifiers. For example, the image processor 212 is configured to classify a cardiac condition of the patient from output of a few-shot machine-trained model adapted from a multi-task trained initial model where multiple tasks of the multi-task trained initial model are anatomical and/or functional characteristics linked to the cardiac condition

The image processor 212 is configured to generate an image. An image showing the predicted classification is generated. The classification may be displayed with an image of the interior of the patient, such as a MR image.

The image processor 212 may be configured to estimate uncertainty for a classification. The uncertainty may be output with the classification.

The display 210 is a CRT, LCD, projector, plasma, printer, tablet, smart phone or other now known or later developed display device for displaying information derived from the output of the model. For example, the display 210 displays an image showing the cardiac condition output by the machine-learned model.

The scan data, training data, medical data, network definitions, features, machine-learned network, and/or other information are stored in a non-transitory computer readable memory, such as the memory 214. The memory 214 is an external storage device, RAM, ROM, database, and/or a local memory (e.g., solid state drive or hard drive). The same or different non-transitory computer readable media may be used for the instructions and other data. The memory 214 may be implemented using a database management system (DBMS) and residing on a memory, such as a hard disk, RAM, or removable media. Alternatively, the memory 214 is internal to the processor 212 (e.g. cache).

The instructions for implementing the training or application processes, the methods, and/or the techniques discussed herein by the processor 212 are provided on non-transitory computer-readable storage media or memories, such as a cache, buffer, RAM, removable media, hard drive or other computer readable storage media (e.g., the memory 214). Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU or system. Because some of the constituent system components and method steps depicted in the accompanying figures may be implemented in software, the actual connections between the system components (or the process steps) may differ depending upon the manner in which the present embodiments are programmed.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A method for disease classification in a medical system, the method comprising:
acquiring a medical scan of a patient;
classifying the disease of the patient from the medical scan, the classifying using input of data from the medical scan to a first machine-learned model having been trained for classification with few-shot learning from a second machine-learned model having been trained for prediction of functional or anatomical characteristics; and
displaying a classification from output by the first machine-learned model in the classifying.

2. The method of claim 1 wherein acquiring comprises acquiring magnetic resonance scan data, and wherein classifying comprises classifying cardiac disease with the first machine-learned model wherein the second machine-learned model was trained for prediction of ejection fraction.

3. The method of claim 1 wherein classifying comprises classifying where the second machine-learned model comprises a multi-task model.

4. The method of claim 1 wherein classifying comprises classifying where the first and second machine-learned models comprise neural networks.

5. The method of claim 1 wherein classifying using the first machine-learned model comprises classifying where the first machine-learned model was trained with the few shot learning where the training used episodes and a long-short term memory.

6. The method of claim 1 wherein classifying comprises classifying where the second machine-learned model was trained with weak supervision as a labeling function.

7. The method of claim 1 further comprising estimating an uncertainty of the classification, and wherein outputting comprises outputting the classification and the uncertainty.

8. The method of claim 1 wherein classifying comprises classifying where the few shot learning included less than 200 samples and where the second machine-learned model was trained with at least 1,000 samples.

9. The method of claim 8 wherein classifying comprises classifying where at least some of the less than 200 samples are synthetic examples.

10. The method of claim 8 wherein classifying comprises classifying where at least some of the at least 1,000 samples are synthetic examples.

11. The method of claim 10 wherein the at least 1,000 samples include a first set of samples from actual people and a second set of samples comprising the synthetic examples, where numbers of values of ground truth provided by the first set of samples has a first variance and wherein the number of values of the ground truth provided by the second set of samples reduces the first variance.

12. The method of claim 1 further comprising generating, by a processor, a clinical decision from the classification.

13. The method of claim 12 further comprising estimating an uncertainty of the classification, and wherein generating comprises generating based on the uncertainty.

14. A method for machine training for disease classification, the method comprising:
identifying an anatomical or functional characteristic linked to a pathology;
locating data samples of patient data having known values of the anatomical and/or functional characteristics;
machine training a first classifier with the data samples as training data where the known values are ground truth, the first classifier machine trained to output the anatomical or functional characteristic;
machine training a second classifier adapted from the machine-trained first classifier, the second classifier machine trained with few-shot learning to output the pathology; and
storing the machine-trained second classifier.

15. The method of claim 14 wherein identifying comprises identifying the anatomical or functional characteristic as ejection fraction and the pathology comprises a type of cardiac disease.

16. The method of claim 14 further comprising generating some of the training data as synthetic samples derived from the data samples.

17. The method of claim 14 wherein machine training the first classifier uses training data with a number of examples at least ten times a number of examples for machine training the second classifier.

18. The method of claim 14 wherein machine training the second classifier comprises machine training where the few-shot learning uses data separation into episodes.

19. The method of claim 14 further comprising predicting an uncertainty of the output of the second classifier based on the machine training of the second classifier.

20. A medical imaging system for cardiac classification, the medical imaging system comprising:
a medical imager configured to scan a patient;
an image processor configured to classify a cardiac condition of the patient from output of a few-shot machine-trained model adapted from a multi-task trained initial model where multiple tasks of the multi-task trained initial model are anatomical and/or functional characteristics linked to the cardiac condition; and
a display configured to display information derived from the cardiac condition.
